(19) United States
(12) Patent Application Publication
(10) Pub. No.: US 2026/0016041 A1
(43) Pub. Date: Jan. 15, 2026

(54) Dual Interface Screw Element and System

(71) Applicant: Yehonathan Unger, Moshav Bustan HaGalil (IL)

(72) Inventor: Yehonathan Unger, Moshav Bustan HaGalil (IL)

(21) Appl. No.: 18/772,435

(22) Filed: Jul. 15, 2024

(51) Int. Cl. F16B 33/02; A61B 17/86; A61C 8/00; F16B 37/12

(52) U.S. Cl. CPC F16B 33/02; F16B 37/125; A61B 17/86; A61C 8/0022; F16B 2033/025

(57) ABSTRACT

A dual interface screw element includes a screw body (10) with an external thread (12) and two distinct engagement interfaces (13, 15), one for inserting the screw and another for removing it. The removal engagement interface (15) includes a reverse thread (16) located in an axial recess (36) such that a complementary reverse-threaded tool (24) applying torque to extract the screw body is tightened against the second engagement interface. The screw is part of a system including an extraction tool (24) and an insertion tool (18). Applications include medical applications such as dental implants and bone screws, domestic applications such as headless wall mounting anchors, and a wide range of other industrial and aerospace applications.

### FIELD AND BACKGROUND OF THE INVENTION

The present invention relates to screws and, in particular, it concerns a screw element and corresponding system with distinct interfaces for inserting and extracting the screw.

Screws are among the most ubiquitous mechanical elements, employed for an unlimited range of applications ranging from medical devices to heavy machinery and from household appliances to aerospace applications.

There are numerous types of screw heads designed to interface with different screwdrivers or driving tools. Common examples include Phillips, flat head, Robertson (square), and Torx. Different head designs offer different advantages and disadvantages, each with its own limitations on what maximum torque can be applied.

A problem common to all screw interfaces is that the screw may disengage or fall from the driver while trying to position it for insertion. This can in some cases be ameliorated by employing a magnetic bit, but his still allows some degree of misalignment between the driver and the screw which may hamper insertion and does not provide a solution for non-magnetic materials.

A further problem relates to unscrewing to remove a screw. In this case, there is an inherent conflict between the strong forward axial force which is required to maintain reliable engagement of a driver with the screw head and the desired direction of motion of the screw which would be enhanced by a rearward extraction force. Furthermore, depending on the materials of the screw and the surrounding material, and on how long the screw has been in place, the torque required for extraction may be larger than the insertion torque, sometimes exceeding the torque which can be delivered through the screw interface.

### SUMMARY OF THE INVENTION

The present invention is a screw element and corresponding system with distinct interfaces for inserting and extracting the screw.

According to the teachings of an embodiment of the present invention there is provided, a dual interface screw element comprising: (a) a screw body having a central axis, a proximal end, a distal end and a length, the screw body having an external thread along at least part of the length; (b) a first engagement interface accessible from the proximal end of the screw body for engagement to supply torque in an insertion direction of the external thread; and (c) a second engagement interface located in an axial recess accessible from the proximal end of the screw body, the second engagement interface including a reverse thread such that a complementary reverse-threaded tool applying torque to extract the screw body is tightened against the second engagement interface.

According to a further feature of an embodiment of the present invention, the external thread is a right-handed thread and wherein the reverse thread is a left-handed thread.

According to a further feature of an embodiment of the present invention, the first engagement interface includes a thread located within the axial recess.

According to a further feature of an embodiment of the present invention, the thread of the first engagement interface is closer to the proximal end than the reverse thread of the second engagement interface.

According to a further feature of an embodiment of the present invention, the thread of the first engagement interface is further from the proximal end than the reverse thread of the second engagement interface.

There is also provided according to the teachings of an embodiment of the present invention, a screw system comprising: (a) the aforementioned screw element; and (b) an extraction tool comprising a reverse-threaded portion configured for insertion into the axial recess and for engaging the second engagement interface.

There is also provided according to the teachings of an embodiment of the present invention, a screw system comprising: (a) the aforementioned screw element; (b) an insertion tool comprising a threaded portion configured for insertion into the axial recess and for engaging the first engagement interface; and (c) an extraction tool comprising a reverse-threaded portion configured for insertion into the axial recess and for engaging the second engagement interface.

According to a further feature of an embodiment of the present invention, the screw body is a dental implant.

According to a further feature of an embodiment of the present invention, the screw body is a bone screw.

According to a further feature of an embodiment of the present invention, the screw body is a bolt forming part of a joint between two structural elements.

There is also provided according to the teachings of an embodiment of the present invention, a dual interface screw element comprising: (a) a screw body having a central axis, a proximal end, a distal end and a length, the screw body having an external thread along at least part of the length; (b) a first engagement interface accessible from the proximal end of the screw body for engagement to supply torque in an insertion direction of the external thread; and (c) a second engagement interface located in an axial recess accessible from the proximal end of the screw body, the second engagement interface including a reverse helical engagement configuration such that a complementary reverse-helical engagement tool applying torque to extract the screw body is tightened against the second engagement interface.

According to a further feature of an embodiment of the present invention, the reverse helical engagement configuration is a reverse screw thread.

According to a further feature of an embodiment of the present invention, the reverse helical engagement configuration is a helical socket of hexagonal cross-section. According to a further feature of an embodiment of the present invention, the reverse helical engagement configuration is a helical socket of star cross-section.

### BRIEF DESCRIPTION OF THE DRAWINGS

The invention is herein described, by way of example only, with reference to the accompanying drawings, wherein:

FIG. 1 is a schematic isometric view of a screw element constructed and operative according to an embodiment of the present invention;

FIG. 2 is an axial cross-sectional view of the screw element of FIG. 1 revealing dual engagement interfaces;

FIG. 3 is a schematic isometric view of an insertion tool for engaging a first engagement interface of the screw element of FIG. 1;

FIG. 4 is a schematic isometric view of a removal tool for engaging a second engagement interface of the screw element of FIG. 1;

FIG. 5 is an axial cross-sectional view showing the insertion tool of FIG. 3 engaged with the screw element of FIG. 1;

FIG. 6 is an axial cross-sectional view showing the removal tool of FIG. 4 engaged with the screw element of FIG. 1;

FIG. 7 is an axial cross-sectional view of a variant implementation of the present invention for use as a dental implant;

FIG. 8 is an axial cross-sectional view of a further variant implementation of the present invention for use as a bone screw;

FIG. 9 is a view similar to FIG. 8 illustrating an alternative implementation of the first engagement interface as a hex socket;

FIG. 10 is a view similar to FIG. 8 illustrating an alternative implementation in which the axial positions of the first and second engagement interfaces are exchanged;

FIG. 11 is a view similar to FIG. 10 illustrating an alternative implementation of the first engagement interface as a hex socket;

FIG. 12 is an isometric view cut-away along an axial plane illustrating an alternative implementation of the screw element of FIG. 1 employing helical engagement configurations with a hexagonal cross-section;

FIGS. 13 and 14 are partial isometric views of a complementary insertion tool and a complementary removal tool, respectively, for use with the screw element of FIG. 12;

FIGS. 15A and 15B are isometric views illustrating a further alternative implementation of the screw element of FIG. 1 employing helical engagement configurations with a star cross-section, shown, respectively, in full view and cut-away along an axial plane; and

FIGS. 16 and 17 are partial isometric views of a complementary insertion tool and a complementary removal tool, respectively, for use with the screw element of FIG. 15A.

### DESCRIPTION OF THE PREFERRED EMBODIMENTS

The present invention is a screw element and corresponding system with distinct interfaces for inserting and extracting the screw.

The principles and operation of screw elements and systems according to the present invention may be better understood with reference to the drawings and the accompanying description.

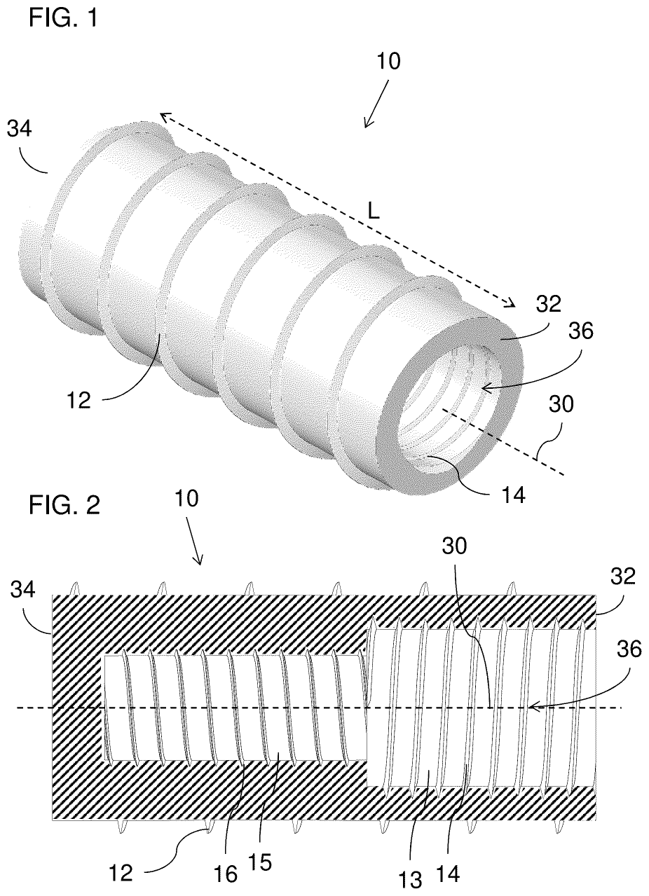

Referring now to the drawings, FIGS. 1 and 2 show schematically a dual interface screw element, constructed and operative according to an embodiment of the present invention. In general terms, the screw element has a screw body 10 having a central axis 30, a proximal end 32, a distal end 34 and a length L. Screw body 10 has an external thread 12 along at least part of the length. The screw element has two distinct engagement interfaces, one designed for use while inserting the screw and another for use when removing (unscrewing) the screw. Thus, a first engagement interface 13, accessible from the proximal end of the screw body, is configured for engagement to supply torque in an insertion direction of the external thread. In the non-limiting but preferred case illustrated here, this first engagement interface 13 includes a thread 14 located within an axial recess 36. A second engagement interface 15, located in axial recess 36 accessible from the proximal end 32 of screw body 10, includes a reverse thread 16 such that a complementary reverse-threaded tool (FIG. 4, below) applying torque to extract the screw body is tightened against the second engagement interface. In the case of a conventional right-handed external thread 12, the “reverse” thread 16 is a left-handed thread. In any special application requiring a left-handed external thread 12, the “reverse” thread 16 would be a right-handed thread.

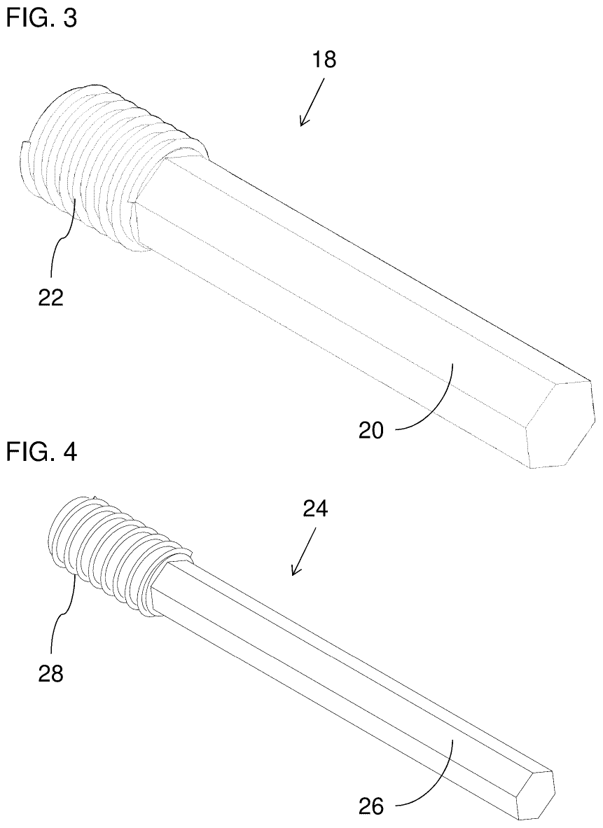

The screw of FIG. 1 is preferably provided as part of a screw system which includes an extraction tool 24 (FIG. 4), having a reverse-threaded portion 28 configured for insertion into the axial recess 36 to engage thread 16 of the second engagement interface 15. The screw system preferably also includes an insertion tool 18 (FIG. 3) having a threaded portion 22 configured for insertion into axial recess 36 to engage thread 14 of the first engagement interface 13. Insertion tool 18 and extraction tool 24 each has a shaft 20 and 26, respectively, for delivering torque to the screw during insertion or extraction. In the non-limiting but preferred case illustrated here, these shafts are hexagonal shafts suitable for mounting in a conventional powered drill-driver or impact driver. Optionally, both tool ends (threaded portions 22 and 28) may be incorporated on opposite ends of a single straight hex shaft to form a reversible tool (not shown), or on opposite ends of an L-shaped shaft to form a manual tool for insertion and extraction that is operated similarly to an Allen key. Alternative implementations may employ a manually operated handle on the shaft.

FIG. 5 illustrates schematically use of insertion tool 18 to insert screw body 10 into a host material (not shown). Threaded portion 22 engages thread 14 within the proximal part of axial recess 36 through a right-handed rotation until it is fully inserted, which may be defined by either a distal part of shaft 20 abutting proximal end 32 of the screw body or by the threaded portion 22 abutting an internal shoulder of recess 36, as illustrated in FIG. 5. In this state, the screw body is firmly gripped and aligned with the insertion tool, preventing accidental dropping or misalignment of the screw. Further right-handed rotation of insertion tool 18 transfers torque to screw body 10 so as to insert the screw body into the host material in a conventional manner.

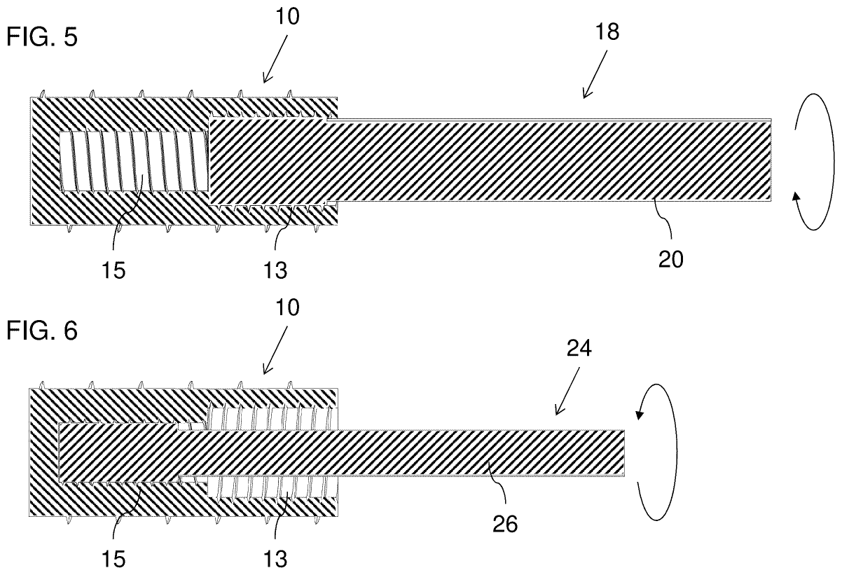

FIG. 6 illustrates schematically use of extraction tool 24 to extract (unscrew) screw body 10 from a host material (not shown). Threaded portion 28 is inserted through the proximal part of axial recess 36, being undersized so as not to interact with thread 14, and then engages internal thread 16 through a left-handed rotation until fully engaged. The fully engaged state may be defined by a suitably positioned and sized shoulder formed on shaft 26 to engage the internal shoulder within recess 36 at the transition between the two engagement configurations, by a larger shoulder configured to abut proximal end 32 of the screw body, or by threaded portion 28 reaching the end of the threaded recess as shown in FIG. 6. In this state, the screw body is firmly gripped by the extraction tool so that further left-handed rotation of extraction tool 24 transfers torque to screw body 10 so as to unscrew the screw body into the host material. The reverse-threaded engagement ensures that the torque applied to unscrew the screw body only tightens engagement of extraction tool 24, and this engagement also facilitates application of axial forces to aid withdrawal of the screw body from its host material.

Optionally, when engaging the smaller diameter engagement interface 15 further from the proximal end of the screw body, a cylindrical spacer element may be introduced in order to provide support for the part of the shaft 26 (or 20 if the interfaces are reversed, as discussed below) which is within the larger diameter part of the recess. In this case, the spacer element is preferably sized to contact the crests of the threads in the larger diameter part of the recess without engaging the threads.

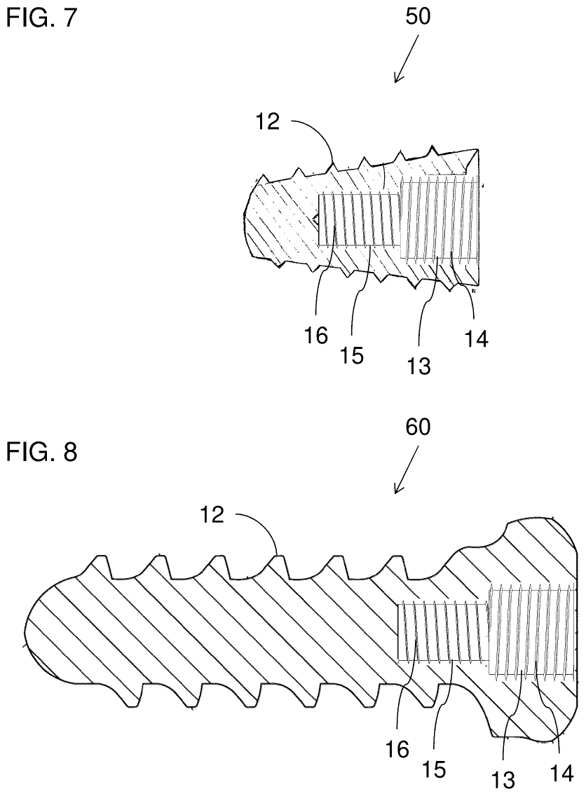

The screw element of FIG. 1 is illustrated only schematically as a cylindrical screw element with an external thread. The present invention may be used to advantage in all fields of application and all types of screw or bolt structures, particularly where large closing torque is required, and where subsequent extraction of a screw may otherwise be challenging. Non-limiting exemplary applications include implementations as a dental implant 50 (FIG. 7) or as a bone screw 60 (FIG. 8). Other applications include domestic and other applications such as, for example, as a headless wall anchor (in which case an object may be supported by engaging screw thread 14 of axial recess 36) and in all sorts of screws and bolts for forming part of a joint between two structural elements, in domestic, industrial and aerospace applications.

Although it is believed to be highly advantageous to implement both first engagement interface 13 and second engagement interface 15 as threaded interfaces (or, more generically, “helical engagement configurations” as discussed further below), it should be noted that screws and screw systems in which the first engagement interface is an otherwise conventional engagement interface also fall within the scope of the present invention. Thus, for example, the first engagement interface may be implemented in recess 36 and/or on the surface of the proximal end 32 of the screw as a straight slot, a Philips crosshead, a Torx star socket, a hex socket or any other conventional or non-standard screw interface. By way of one non-limiting example, FIG. 9 illustrates an exemplary embodiment in which first engagement interface 13 is implemented as a hex socket.

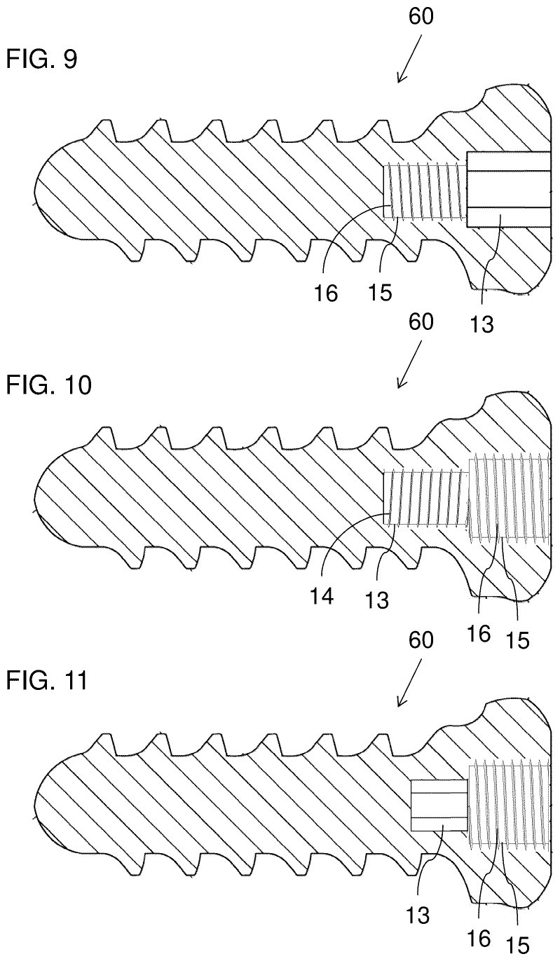

The exemplary implementations of the present invention illustrated thus far all have first engagement interface 13 closer to proximal end 32 than second engagement interface 15. Depending on various design considerations, and most notably, the levels of torque for which the screw system is designed during the insertion and extraction processes, the interfaces may be reversed so that thread 14 of first engagement interface 13 is further from the proximal end 32 than reverse thread 16 of second engagement interface 15. This option is illustrated in FIG. 10. A further variant implementation, generally similar to FIG. 10 but in which first engagement interface 13 is implemented as a hex socket, is illustrated in FIG. 11.

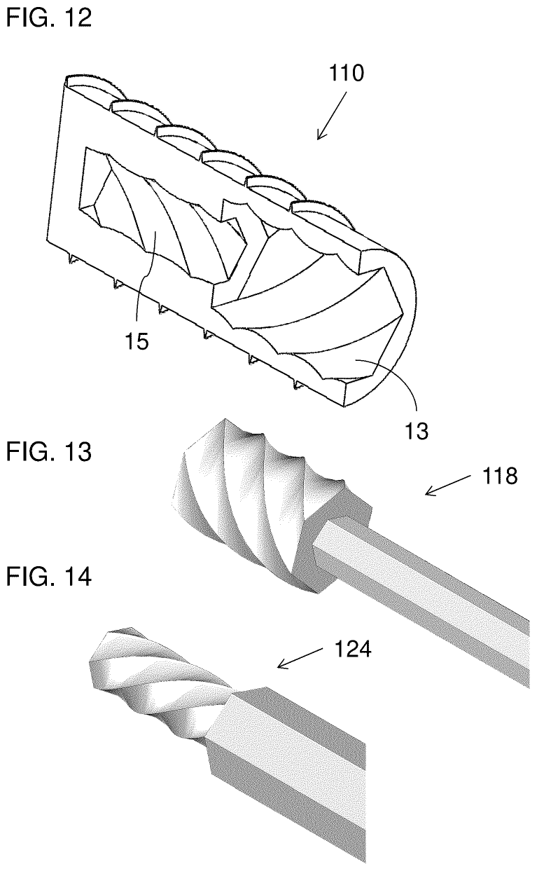

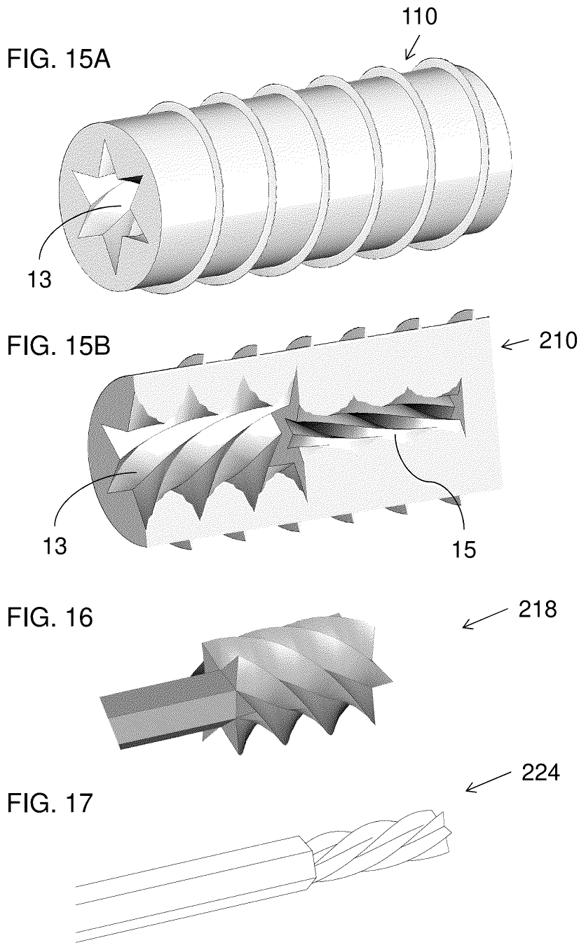

The threaded engagement of first and second engagement interfaces illustrated thus far are examples of a “helical engagement configuration,” i.e., any complementary male-female engagement configuration in which engagement is achieved by a combination of simultaneous motion along an axis combined with rotation about that axis. Depending on details of the intended application, material properties and manufacturing capabilities, other forms of helical engagement configuration may be preferred for one or both of first engagement interface 13 and second engagement interface 15, and the complementary insertion and extraction tools. By way of non-limiting examples, FIGS. 12-14 illustrate an embodiment of the screw element 110, insertion tool 118 and extraction tool 124, respectively, where each engagement interface is implemented as a helical engagement element which has a hexagonal cross-section perpendicular to its central axis at each point, and FIGS. 15A-17 illustrate an embodiment of the screw element 210, insertion tool 218 and extraction tool 224, respectively, where each engagement interface is implemented as a helical engagement element which has a star cross-section perpendicular to its central axis at each point. Here too, the various engagement interfaces may be of different types, the inner and outer configurations may be swapped axially, and first engagement interface may be replaced with an otherwise conventional interface.

It will be appreciated that the above descriptions are intended only to serve as examples, and that many other embodiments are possible within the scope of the present invention as defined in the appended claims.

What is claimed is:

1. A dual interface screw element comprising:
(a) a screw body having a central axis, a proximal end, a distal end and a length, said screw body having an external thread along at least part of the length;
(b) a first engagement interface accessible from said proximal end of said screw body for engagement to supply torque in an insertion direction of said external thread; and
(c) a second engagement interface located in an axial recess accessible from said proximal end of said screw body, said second engagement interface including a reverse thread such that a complementary reverse-threaded tool applying torque to extract said screw body is tightened against said second engagement interface.

2. The screw element of claim 1, wherein said external thread is a right-handed thread and wherein said reverse thread is a left-handed thread.

3. The screw element of claim 1, wherein said first engagement interface includes a thread located within said axial recess.

4. The screw element of claim 3, wherein said thread of said first engagement interface is closer to said proximal end than said reverse thread of said second engagement interface.

5. The screw element of claim 3, wherein said thread of said first engagement interface is further from said proximal end than said reverse thread of said second engagement interface.

6. A screw system comprising:
(a) the screw element of claim 1; and
(b) an extraction tool comprising a reverse-threaded portion configured for insertion into said axial recess and for engaging said second engagement interface.

7. A screw system comprising:
(a) the screw element of claim 3;
(b) an insertion tool comprising a threaded portion configured for insertion into said axial recess and for engaging said first engagement interface; and
(c) an extraction tool comprising a reverse-threaded portion configured for insertion into said axial recess and for engaging said second engagement interface.

8. The screw element of claim 1, wherein said screw body is a dental implant.

9. The screw element of claim 1, wherein said screw body is a bone screw.

10. The screw element of claim 1, wherein said screw body is a bolt forming part of a joint between two structural elements.

11. A dual interface screw element comprising:
(a) a screw body having a central axis, a proximal end, a distal end and a length, said screw body having an external thread along at least part of the length;
(b) a first engagement interface accessible from said proximal end of said screw body for engagement to supply torque in an insertion direction of said external thread; and
(c) a second engagement interface located in an axial recess accessible from said proximal end of said screw body, said second engagement interface including a reverse helical engagement configuration such that a complementary reverse-helical engagement tool applying torque to extract said screw body is tightened against said second engagement interface.

12. The screw element of claim 11, wherein said reverse helical engagement configuration is a reverse screw thread.

13. The screw element of claim 11, wherein said reverse helical engagement configuration is a helical socket of hexagonal cross-section.

14. The screw element of claim 11, wherein said reverse helical engagement configuration is a helical socket of star cross-section.
